(19) United States
(12) Patent Application Publication
(10) Pub. No.: US 2022/0062144 A1
(43) Pub. Date: Mar. 3, 2022

(54) HAIR AND SKIN TREATMENT

(71) Applicant: SES Research Inc., Houston, TX (US)

(72) Inventors: Christopher V. Burres, Houston, TX (US); Robert Wong, Houston, TX (US); Fabio Rumbao Pedreira, Houston, TX (US)

(21) Appl. No.: 17/231,607

(22) Filed: Apr. 15, 2021

Related U.S. Application Data
(60) Provisional application No. 63/070,099, filed on Aug. 25, 2020.

(51) Int. Cl. A61K 8/64; A61K 8/19; A61Q 7/00; A61K 8/49; A61K 8/9794; A61K 8/92; A61Q 19/00

(52) U.S. Cl. CPC A61K 8/64; A61K 8/19; A61Q 7/00; A61K 2800/874; A61K 8/9794; A61K 8/922; A61Q 19/00; A61K 8/4953

(57) ABSTRACT

Formulations for promoting hair growth, preventing or mitigating hair loss, or enhancing skincare include a GHK peptide and a fullerene, such as C₆₀, that has been processed to be safe for human consumption. In examples, the fullerene includes ESS60. In examples, the GHK peptide includes a GHK-Cu peptide, such as UTH29. Such therapeutic formulations may be applied to a person's skin or hair. In some embodiments, the peptide is dissolved in a polar solvent. In some embodiments, the fullerene is dissolved in a lipid solution. The formulations, when administered topically or introduced into the top layers of the epidermis, are effective in preventing or mitigating hair loss and graying that arises from age or elevated DHT levels. The formulations are also effective for use in the lightening of skin blemishes and prevention or mitigation of certain skin cancers that arise from age and UV damage.

### CROSS-REFERENCE TO RELATED APPLICATION

This application claims priority to U.S. Provisional Patent Application Ser. No. 63/070,099 filed Aug. 25, 2020, the contents of which are incorporated herein by reference.

### BACKGROUND

The present disclosure generally relates to a formulation for hair growth, hair loss prevention, hair treatment, and/or skincare, including the treatment of wrinkles, liver spots, and/or sun damage.

Hair loss afflicts numerous male and female individuals. Hair loss includes several types such as alopecia areata, male-pattern hair loss, female-pattern hair loss, and hair thinning. Hair loss may also refer to a loss of hair coloration, resulting in graying or whitening of the hair. Due to protective benefits and substantial aesthetic benefits of a full head of hair, numerous individuals seek treatment to prevent, slow, or reverse hair loss.

Treatments available for hair loss are numerous and include various procedures and drugs. A large industry has arisen that purport to treat hair loss; however, there are a large number of hair growth treatments that are unproven or produce unsatisfactory results. Some treatments result in undesirable side effects.

As a person ages, the skin loses elasticity and can become damaged from exposure to ultraviolet light, toxins, and other harmful causes. Wrinkles, liver spots, mottled pigmentation, and other skin blemishes may appear on affected skin. Many people seek to prevent, mitigate, and/or reverse such blemishes in attempts to appear younger and to have healthier skin. However, as in the hair treatment industry, there are a multitude of unproven and ineffective “anti-aging” skincare products.

### DETAILED DESCRIPTION

Before explaining the present process in detail, it is to be understood that the process is not limited to the particular embodiments and that it can be practiced or carried out in various ways.

Embodiments of the present disclosure relate to formulations that can be used as a topical treatment for promoting hair growth, restoring hair coloration, preventing or mitigating hair loss, and/or for skincare. In the present disclosure, a skincare treatment may further refer to promoting hair growth, such as by reversing hair loss and/or causing the growth of hair where there was previously no hair or thinner hair than desired, to preventing or mitigating hair loss, or to preventing or mitigating loss of hair color. Skincare treatments may further refer to restoring hair color from changes associated with aging, including changing the hair back to youthful-appearing hair, including but not limited to restoring hair that has greyed or whitened as a person aged.

Embodiments comprise a fullerene and a glycyl-l-histidyl-l-lysine (“GHK”) peptide. Such compositions may include carriers and/or diluents. The treatments may be administered by direct topical application to the area where increased hair growth and/or restoration is preferred, for example on a user's scalp area or on other areas of the user's skin. In some embodiments, a composition as set forth in herein is applied to a user's scalp as an anticipatory measure in order to prevent and/or mitigate hair loss. Other methods of administration may be employed.

According to various embodiments, the fullerene of the present disclosure may comprise a C₆₀ compound, also known as a buckminsterfullerene. In embodiments, the fullerene is dissolved in a lipid solution. In such a solution, the solvent may include one or more of olive oil, a medium-chain triglyceride (“MCT”) oil, avocado oil, sunflower seed oil, hemp oil, vegetable oil, corn oil, palm oil, fish oil, peanut oil, grapeseed oil, walnut oil, pumpkin seed oil, ghee, and/or blackseed oil. The fullerene may have a concentration in the lipid solution between 0.01% and 5%.

In some embodiments, the fullerene comprises C₆₀ that has been processed to be safe for human consumption. Such a fullerene may be prepared by methods currently known in the art. As would be understood by a person of ordinary skill in the art having the benefit of this disclosure, a process for making C₆₀ safe for human consumption may comprise removing or reducing volatile organic compounds (“VOCs”) from a composition of fullerenes.

In some embodiments, a buffer carrier gas may be employed to remove or reduce VOCs from a composition of fullerenes, thereby resulting in C₆₀ that is safe for human consumption. In some embodiments, C₆₀ may be made safe for human consumption through a vacuum-oven baking process that removes or reduces VOCs from the C₆₀.

In other embodiments, C₆₀ may be manufactured and/or extracted via a process that does not substantially expose the C₆₀ compound to VOCs, in which case the C₆₀ may be considered safe for human consumption. In one such embodiment, C₆₀ is prepared in a sublimation process. One example of C₆₀ that has been processed to be safe for human consumption is sold under the trade name ESS60 by SES Research Inc. of Houston, Tex.

In some embodiments, the fullerene is adapted to be water-soluble, for example by synthesizing the fullerene into a water-soluble fullerene derivative. In these embodiments, the adapted fullerene is dissolved in water at a concentration between 0.01% and 5%. Some embodiments comprise C₆₀ that is adapted to be water-soluble. In these embodiments, the adapted C₆₀ is dissolved in water at a concentration between 0.01% and 5%.

According to various embodiments, the GHK peptide is dissolved in a polar solvent. For example, one embodiment comprises GHK peptide dissolved in water. In another example, one embodiment comprises GHK peptide dissolved in aloe vera juice and/or gel. The GHK peptide may be dissolved in a polar solvent at various concentrations. In some embodiments, the GHK peptide is dissolved in a polar solvent at a concentration between 0.01% and 5%.

In some embodiments, the GHK peptide comprises GHK-copper (“GHK-Cu”) peptide. The GHK-Cu complex may be dissolved in a polar solvent. In various embodiments, this polar solvent comprises water. In other embodiments, this polar solvent comprises aloe vera juice and/or gel. In other embodiments, this polar solvent comprises a combination of water, aloe vera juice, and/or aloe vera gel.

According to various embodiments, the GHK-Cu complex has a concentration between 0.01% and 5% in the polar solvent. In some embodiments, the GHK-Cu complex has a concentration in the polar solvent greater than 5%. For example, some embodiments comprise a GHK-Cu complex having a concentration between 0.01% and 10% in the polar solvent.

In some embodiments, the GHK peptide comprises GHK-Cu that has been processed to be safe for human consumption. Such a GHK peptide may be prepared by methods currently known in the art. As would be understood by a person of ordinary skill in the art having the benefit of this disclosure, a process for making GHK peptide safe for human consumption may comprise removing or reducing VOCs from a composition of GHK peptide.

In other embodiments, a GHK peptide may be manufactured and/or extracted via a process that does not substantially expose the GHK peptide compound to VOCs, in which case the GHK peptide may be considered safe for human consumption. One example of GHK-Cu peptide that has been processed to be safe for human consumption is sold under the trade name UTH29 by SES Research Inc. of Houston, Texas.

Compositions of the present disclosure may be prepared by various processes. In various embodiments, ingredients are mixed at room temperature (approximately 68-72 degrees Fahrenheit) in some embodiments, ingredients are mixed at a temperature between approximately 50-90 degrees Fahrenheit. In various embodiments, ingredients are mixed at a temperature high enough that the ingredients are not below their respective freezing points and low enough that the ingredients are not above their respective boiling points. In the present disclosure, the term “approximately,” with respect to temperature, means within 5 degrees Fahrenheit of the stated value.

According to various embodiments of the present disclosure, compositions may be prepared by mixing ingredients at atmospheric pressure (approximately 14.7 psi). In some embodiments, ingredients are mixed at a pressure between approximately 10-20 psi. In other embodiments, ingredients are mixed at lower or higher-pressure ranges than disclosed herein. In the present disclosure, the term “approximately,” with respect to pressure, means within 2 psi of the stated value.

As set forth above, embodiments of the present disclosure comprise mixing a fullerene with a lipid solution. According to one embodiment, a lipid and a fullerene are mixed for a time of approximately twenty-one days. In some cases, mixing may be complete once the lipid/fullerene mixture is saturated and/or homogeneous. In various embodiments, any type of container and/or mixing apparatus may be utilized to mix the fullerene with the lipid. In one embodiment, the lipid and the fullerene are mixed in an Erlenmeyer flask using a magnetic stir bar. In other embodiments, the lipid and the fullerene are mixed in various types of industrial mixers.

As set forth above, embodiments of the present disclosure comprise mixing a peptide with a polar solvent. According to one embodiment, a peptide and a polar solvent are mixed for approximately 20 minutes. In other embodiments, the peptide and the polar solvent are mixed until homogenous. In various embodiments, any type of container and/or mixing apparatus may be utilized to mix the peptide with the polar solvent. In one embodiment, the peptide and the polar solvent are mixed in an Erlenmeyer flask using a magnetic stir bar. In other embodiments, the peptide with the polar solvent is mixed in various types of industrial mixers.

In some embodiments of the present disclosure, a buffer gas is injected into a mixing chamber to prevent or mitigate oxidation of the ingredients being mixed. In some embodiments, mixing is carried out by sonication. Sonication may be used alone or in conjunction with mechanical mixing operations. In the present disclosure, the term “approximately,” with respect to time, means within 5% of the stated value.

In the present disclosure, the term “concentration” refers to a weight/volume percent concentration, which is defined as a solute mass divided by a solution volume, expressed as a percentage. For example, a solution having a concentration of 5% GHK-CU in water may be prepared by mixing 5 grams GHK-CU in 100 mL water.

Some embodiments of the present disclosure comprise skincare, hair care, or similar compositions that are offered to retail customers as a mixture comprising a fullerene/lipid composition mixed with a peptide/polar solvent composition. As would be understood by a person of ordinary skill in the art having the benefit of this disclosure, such compositions comprising a lipid and a polar solvent may separate out by the time the end user is ready to apply the composition. Thus, in some embodiments, a fullerene/lipid composition and a peptide/polar solvent composition may be offered separately, in multiple containers to retail customers. Such multiple compositions may be accompanied by instructions for the end user to combine and mix the multiple compositions prior to application. For example, the user may mix multiple compositions in-hand prior to application on the skin. Other embodiments comprise an emulsifier or other additive that may prevent or mitigate separation of the compositions; such embodiments may include pre-mixing the multiple compositions prior to final distribution and/or sale.

The compositions described above may be used in numerous types of applications, including but not limited to various topical ointments. Embodiments of the present disclosure include eye serum (applied to the skin below the eye), nighttime skin serum, daytime skin serum, suntan lotion, leave-in hair conditioner, general body lotion, lip balm, lip moisturizer, other lip products, wrinkle filler, anti-aging cream, skin lightener/whitener, skin blemish remover, preventative acne ointment, acne scar treatment ointment, liver spot treatment, eczema treatment, rash ointment, and other compositions for application to the skin and/or hair. Some embodiments of the present disclosure may be used to treat applied to multiple conditions and/or in various of the foregoing applications.

In one embodiment, application of compositions disclosed herein to the user's skin is made by use of a skin roller apparatus. In embodiments, a skin roller may be a derma roller, a microneedle roller, and/or a jade roller. According to some embodiments, a derma roller or a microneedle roller comprises numerous needles on a rolling surface that puncture the user's skin as the rolling surface rotates and rolls across the skin. According to various embodiments, a jade roller comprises a massaging roller made of jade, stone, or other materials.

In some embodiments, a user may roll a skin roller across the skin and/or scalp to pre-treat the skin by massaging and/or needling the skin surface prior to application of compositions described herein to the pre-treated skin. Following this pre-treatment, one or more of the various compositions described herein may be applied to and worked into the skin. In many cases, the compositions are worked into the skin by rubbing the skin with the hands after depositing a desired amount of the composition at a targeted area. In some cases, the composition is worked into the skin by rubbing the skin with various types of applicator pads or devices. In other embodiments, a user may apply a composition to the skin and then roll the skin roller across the skin to work the composition into the skin while massaging and/or needling the skin surface.

According to various embodiments, the composition to be applied in conjunction with a skin roller may be a fullerene composition as described above, a peptide composition as described above, or a combination of fullerene and peptide compositions as described above.

In some embodiments, an ultrasonic skin spatula, skin scrubber, or other electromechanical vibration device is used to pre-treat the skin and/or scalp prior to application of a treatment formulation. An example skin scrubber vibrates a pad at approximately 24 kHz as the pad is applied to the user's skin.

As set forth above, some embodiments of the present disclosure include GHK-Cu peptide in various concentrations. In such embodiments that are prepared for use as an eye serum, GHK-Cu peptide may be included at a relatively lower concentration in comparison to other formulations disclosed herein. A lower concentration of GHK-Cu peptide may reduce the risk of irritation to the eye.

As used herein, the term “providing” means furnishing, supplying, making available, or preparing, in whole or in multiple parts, or all at once or over a period of time.

Some embodiments of the present disclosure comprise a fullerene and a GHK peptide as set forth above, and additionally include minoxidil. As would be understood by a person of ordinary skill in the art having the benefit of this disclosure, minoxidil may be used to stimulate hair-growth and/or mitigate hair loss.

While these embodiments have been described with emphasis on the embodiments disclosed, it should be understood that within the scope of the appended claims, the embodiments might be practiced other than as specifically described herein.

What is claimed is:

1. A composition comprising:
a fullerene
a glycyl-l-histidyl-l-lysine (“GHK”) peptide.

2. The composition of claim 1, wherein the fullerene comprises C₆₀.

3. The composition of claim 2, wherein the C₆₀ has been processed to be safe for human consumption.

4. The composition of claim 2, wherein the C₆₀ is dissolved in a lipid solution.

5. The composition of claim 4, wherein the lipid is selected from the group consisting of: olive oil, MCT oil, avocado oil, sunflower seed oil, hemp oil, vegetable oil, corn oil, palm oil, fish oil, peanut oil, grapeseed oil, walnut oil, pumpkin seed oil, ghee, and blackseed oil.

6. The composition of claim 4, wherein the C₆₀ has a concentration in the lipid solution between 0.01% and 5%.

7. The composition of claim 2, wherein:
the C₆₀ is adapted to be water-soluble; and
the C₆₀ is dissolved in water solution at a concentration between 0.01% and 5%.

8. The composition of claim 1, wherein the GHK peptide is dissolved in a polar solution.

9. The composition of claim 8, wherein the GHK polar solution comprises water or aloe vera juice.

10. The composition of claim 1, wherein the GHK peptide comprises a GHK-copper (“GHK-Cu”) peptide.

11. The composition of claim 10, wherein the GHK-Cu peptide is dissolved in a polar solvent at a concentration between 0.01% and 5%.

12. The composition of claim 1, further comprising minoxidil.

13. A method of applying a treatment, comprising:
providing a hair growth treatment composition, the composition comprising:
a fullerene and
a glycyl-l-histidyl-l-lysine (“GHK”) peptide;
applying the hair growth treatment composition to a user's skin or scalp.

14. The method of claim 13, wherein the hair growth treatment composition is used to treat male pattern baldness or female pattern baldness.

15. The method of claim 13, further comprising pretreating the user's skin or scalp with a roller or a skin scrubber.

16. The method of claim 13, wherein the hair growth treatment composition is used to treat androgenetic alopecia.

17. The method of claim 13, wherein the user's skin comprises the user's scalp.

18. A method of applying a skincare treatment, comprising:
providing a skincare treatment composition, the composition comprising:
a fullerene and
a glycyl-l-histidyl-l-lysine (“GHK”) peptide;
applying the skincare treatment composition to a skin condition on a user's skin.

19. The method of claim 18, wherein the skin condition is acne.

20. The method of claim 18, wherein the fullerene comprises C₆₀ and the GHK peptide comprises a GHK-copper (“GHK-Cu”) peptide, further comprising:
dissolving the fullerene in a lipid solution;
dissolving the GHK peptide in a polar solvent; and
mixing the lipid solution and the polar solvent together.
